(19) United States
(12) Patent Application Publication
(10) Pub. No.: US 2023/0366798 A1
(43) Pub. Date: Nov. 16, 2023

(54) ANALYTICAL APPARATUS

(71) Applicant: Stanhope Seta, Chertsey, Surrey (GB)

(72) Inventors: Hatim Thayyil, Chertsey, Surrey (GB); Ian Mylrea, Chertsey, Surrey (GB)

(73) Assignee: Stanhope Seta, Chertsey, Surrey (GB)

(21) Appl. No.: 18/029,187

(22) PCT Filed: Sep. 29, 2021
(86) PCT No.: PCT/GB2021/000110

§ 371 Date: Mar. 29, 2023

(30) Foreign Application Priority Data
Sep. 30, 2020 (GB) 2015513.1

(51) Int. Cl. G01N 9/00; G01N 11/10; G01N 27/06

(52) U.S. Cl. CPC G01N 9/002; G01N 11/10; G01N 27/06

(57) ABSTRACT

An apparatus for simultaneously measuring permittivity, density and temperature of a fluid test sample. The apparatus includes a solid state temperature control means for conditioning the fluid test sample temperature in-situ and a test cell for receiving the fluid test sample. The test cell includes a density sensor, a permittivity sensor, and a temperature sensor. The apparatus further includes a controller communicably coupled to the density sensor, the permittivity sensor, the temperature sensor and the solid state temperature control means.

The present invention relates to laboratory apparatus for the simultaneous measurement of permittivity, density and temperature. More particularly, the present invention relates to laboratory apparatus which may be automated, miniaturized, integrated, and/or self-contained which allows simultaneous measurement of permittivity, density and temperature of a fluid test sample.

The permittivity of a material is a measurement of the polarizability of a material. Permittivity provides an indication of how easily a material can become polarized by the imposition of an electric field upon the material. The polarization of a material sample is an essential physical phenomenon that is useful in various areas. Therefore, permittivity measurement, control and characterisation are critical in many applications. Permittivity is used in aircraft gauging systems to estimate the density and to calculate the mass of fuel remaining in a tank. Traditional direct measurements of the density or mass of a fluid may be unreliable at altitude due to the difficulties posed by physical and/or physiochemical effects such as off-gassing. While a relationship between the density and permittivity of a fluid is known to exist, the relationship is not derivable from first principles and so is subject to inaccuracies and/or approximations. Historically the aviation industry has relied upon the use of data tables which document the characteristics and properties of standard aviation fuels. However, recent developments in new synthetic aviation fuels have provided a range of fuels with different properties which may affect the accuracy of aviation fuel gauging systems. The measurement of permittivity is not limited to aviation and is important in other sectors such as fundamental scientific research, adhesives, paints, lubricants, and oils for industrial scale equipment. Permittivity measurement is not limited to the applications described herein and may be applicable to any suitable application.

The relative permittivity and volumetric mass density of a sample are required to determine the total polarisation in a sample material. Both relative permittivity and volumetric mass density are temperature dependent and there is a relationship between them. This relationship is often difficult to measure and there are multiple theoretical equations relating them. For non-polar and linear gases, the relationship is approximated by the Clausius-Mossotti equation. In addition there are various theoretical equations that approximate the relationship between the volumetric mass density of a liquid sample and the relative permittivity. The Debye equations are satisfactory for some liquids with small amounts of polar substances. To approximate the relationship for polar liquids, the Onsanger-Kirkwood equations may be used. By way of example the Clausius-Mossotti equation is shown in equation 1. In equation 1, εᵣ is the dielectric constant of a material, ε₀ is the permittivity of free space, N is the number density of the molecules of the material typically expressed as a number per m³, and a is the molecular polarizability typically expressed in units of C·m²/V.

Equation 1:

$$\frac{{ε}_{r} - 1}{{ε}_{r} + 2} = \frac{N α}{3 {ε}_{0}}$$

Normal analytical practice involves the measurement of permittivity and density on separate devices. However, even when separate measurements are taken on specialised apparatus the permittivity measurement is not well standardised. Consequently, measurements of permittivity and the relationship with density may have high variability, poor accuracy and require a significant amount of time to measure. Moreover, many industry standard test methods used in various fields require that the permittivity and density of a material be measured independently using separate apparatus. Industry standards may therefore perpetuate and/or propagate the shortcomings of current analytical preferences across and/or between industries.

There are several disadvantages to the current methodologies and known devices for measurement of both permittivity and density. In general, established methods use separate devices to measure each variable and such devices may: require a long time to reach a state of thermal equilibrium; require a large amount of space for installation and operation of the apparatus and its associated components; have high purchase and installation costs; have high ongoing operating costs; and/or necessitate the use of significant labour time. Large samples are also often required for permittivity measurement in known apparatus. Air and moisture may also affect the measurement of permittivity and such measurements rely upon accurate calibration of equipment with a vacuum. For the avoidance of doubt, a vacuum as described herein is considered to be an environment of <2 kPa. Calibration with a vacuum is not easily achieved in apparatus using the thermal baths, environmental chambers or forced-draft ovens which have been adopted previously to address one or more of the shortcomings of established permittivity and density measurement devices. In established devices where thermal baths are used, such baths often contain a flammable liquid such as heptane which is undesirable from both practical and safety perspectives. Such solutions often require manual intervention from a user throughout a test method and may be associated with further issues. In effect, the measurement of permittivity using established methods and apparatus is in the domain of the academic scientist who is able to correctly set up and calibrate disparate or complex apparatus with limited methodologies available. There is therefore a need for analytical apparatus and methods that address the shortcomings of established analytical equipment and methods that may be used easily and reliably in an industrial environment.

The present invention generally relates to an improved apparatus and associated methods for measuring both the permittivity and the density of a dielectric sample using a single apparatus at a constant temperature or range of temperatures. More particularly, the present invention relates to measuring the temperature characteristic of the relative permittivity and the volumetric mass density in a manner which may be automated and/or simultaneous.

According to one aspect of the invention, there is provided an apparatus for simultaneously measuring permittivity, density and temperature of a fluid test sample. The apparatus includes a solid state temperature control means for conditioning the fluid test sample temperature in-situ, a test cell for receiving the fluid test sample, the test cell including a density sensor, a permittivity sensor, and a temperature sensor; and a controller communicably coupled to the density sensor, the permittivity sensor, the temperature sensor and the solid state temperature control means. The solid state temperature control means may include one or more thermoelectric coolers. The solid state temperature control means may include one or more heat sinks. The controller may be configured to automatically adjust the temperature of the test cell towards a predetermined value in response to a signal from the density sensor, the permittivity sensor and/or the temperature sensor.

The controller may be configured to automatically adjust the temperature by sending one or more signals to the solid state temperature control means. The apparatus may further include a reservoir in fluid communication with the test cell. The reservoir may be positioned to receive fluid exceeding the volume of the test cell due to thermal expansion of the fluid test sample when resident in the test cell. The reservoir may include an overflow pipe configured to allow excess fluid to flow out of the reservoir. The apparatus may include an inlet, an outlet, a pump, and one or more valves, wherein the inlet, the outlet, the pump and/or the one or more valves may be configured such that the fluid test sample may be loaded into the test cell via the inlet and subsequently drained from the test cell via the outlet. The controller may be communicably coupled to the pump and/or the one or more valves. The controller may be configured to automatically load the fluid test sample into the test cell and subsequently drain the fluid test sample from the test cell by operating the pump and/or the one or more valves by sending a signal to the pump and/or the one or more valves. The apparatus may include one or more level sensors, and the controller may be configured to actuate the one or more valves and/or operate the pump in response to a signal from the one or more level sensors. The controller may store instructions which, when executed, cause the controller to automatically calculate the relative permittivity of a sample in a test cell, the relationship between permittivity and density, or any combination thereof based on a signal from the density sensor, the permittivity sensor and/or the temperature sensor. The apparatus may further include one or more additional density sensors, permittivity sensors, and/or temperature sensors. Where the apparatus includes one or more additional temperature sensors, the one or more additional temperature sensors may be communicably coupled to the controller, and the controller may be configured to determine a temperature gradient in the test cell based upon a signal from the temperature sensor and the one or more additional temperature sensors and to automatically adjust the temperature of the test cell towards a predetermined value in response the determination of the temperature gradient. The controller stores instructions which, when executed, cause the controller to extrapolate permittivity, relative permittivity, density, temperature, or any combination thereof based upon signals from the density sensor, the permittivity sensor, the temperature sensor and/or any other sensors communicably coupled to the controller. The apparatus may include a display. The controller may be communicably coupled to the display and the controller may be configured such that one or more signals may be sent to the display which cause the display to output information relating to the permittivity, relative permittivity, density, temperature, or any combination thereof of the fluid test sample. The apparatus may include a thermally insulated housing. The solid state temperature control means and/or test cell may be at least partially positioned inside the thermally insulated housing. The permittivity sensor may include a plurality of electrodes. The plurality of electrodes may include a first electrode and a second electrode. The plurality of electrodes may include a first guard electrode and a second guard electrode. The permittivity sensor may include one or more insulating materials. The density sensor may include a flexural resonator. Where the density sensor includes a flexural resonator, the flexural resonator may include one or more U-shaped tubes. The flexural resonator may include a MEMS device. The flexural resonator may be configured to measure viscosity of the fluid test sample based upon the energy lost during oscillation. The controller may be configured to correct a density measurement from the density sensor based upon the measurement of viscosity. The permittivity sensor may be configured to measure the conductivity of a fluid test sample, wherein the conductivity is measured simultaneously alongside a simultaneous measurement of density, permittivity and temperature. The apparatus may be in-line with, or in proximity to, a fluid vessel and configured for continuous or continual simultaneous measurement of permittivity, density and temperature of at least a portion of fluid in the fluid vessel. The fluid vessel may be a pipeline, a fuel-line, a tank, a tank farm, or a fuel bowser.

These and other aspects of the present invention will now be described with reference to the following drawings, in which:

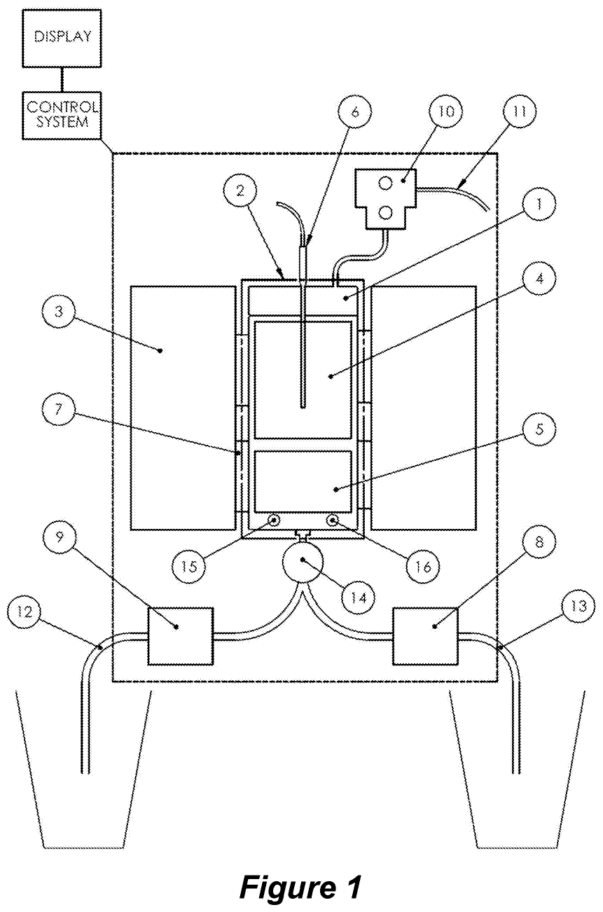

FIG. 1 shows a schematic representation of an analytical apparatus; and

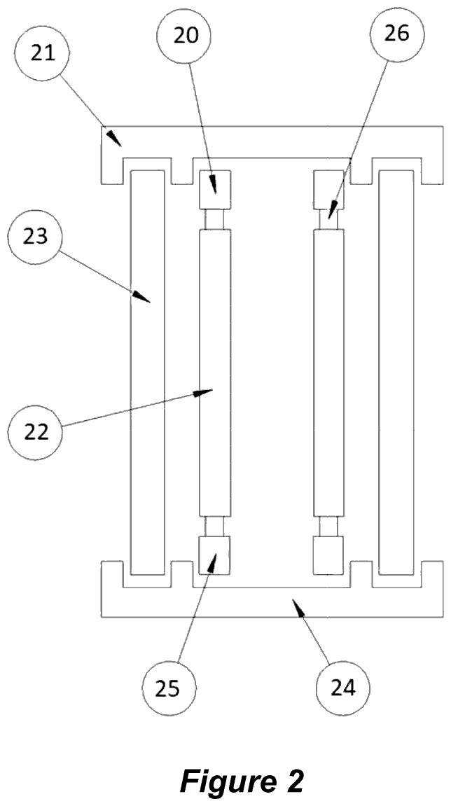

FIG. 2 shows a schematic representation of a permittivity sensor that may be used with the analytical apparatus of FIG. 1.

Apparatus for simultaneously measuring permittivity, density and temperature of a sample will generally include a density sensor, a permittivity sensor, and a temperature sensor forming part of, or proximate to, one or more test cells in which samples to be tested are received. For the avoidance of doubt, the term ‘simultaneously’ as used herein is intended to include truly simultaneous measurement in addition to measurements performed in such close chronological succession so as to be effectively simultaneous. For example, a delay involved in computer processing of approximately 2 seconds between measurements is considered to represent simultaneous measurement. The samples tested using the apparatus will generally be liquid test samples, although any suitable sample including gaseous samples capable of having the properties of permittivity, density and/or temperature measured using the density sensor, permittivity sensor; and/or temperature sensor may be analysed using the apparatus. In one example, the apparatus may be used to measure the permittivity, density, and/or temperature of liquid hydrocarbonaceous samples. Examples of hydrocarbonaceous liquids that may be analysed using the apparatus include fuels, oils, lubricants, isolated hydrocarbon fractions, or any other suitable hydrocarbonaceous liquid. In one particular example, the apparatus may be used to measure the permittivity, density and/or temperature of aviation fuel. In another example, the apparatus may be used to measure the permittivity, density and/or temperature of transformer oils. Although examples of primarily hydrocarbonaceous materials are provided, samples analysed by the apparatus need not be wholly or partly hydrocarbonaceous and may include other species such as diluents, additives, and/or other species.

The test cell may be any suitable receptacle in which a sample may be suitably contained for analysis using the apparatus. The test cell may be any suitable volume depending upon the volume of sample required for analysis by the permittivity sensor, density sensor and/or temperature sensor. In an example, the test cell may have a volume of between 20 ml and 200 ml. In another example, the test cell may have a volume of between 25 ml and 100 ml. In a particular example, the test cell may have a volume of 50 ml. In another particular example, the test cell may have a volume of 75 ml. It may be advantageous for the test cell to have a volume equivalent to the minimum volume of sample required to measure permittivity, density and/or temperature. The test cell may be integral and/or form part of the apparatus in use, or may be removable from the apparatus such that a sample can be loaded into the test cell prior to insertion of the test cell into the apparatus. While the examples provided describe integrated and removable test cells, respectively, the skilled person will understand that a test cell may be semi-integrated such that it is fitted to, and forms part of, the apparatus during general use but may be removed independently for purposes such as maintenance and cleaning. Where the test cell is integral to or forms part of the apparatus, the apparatus may include one or more means by which a sample may be loaded into the test cell, and subsequently removed from the test cell without removal of the test cell from the apparatus. In an example, the apparatus may include one or more fluid conduits, pipes, tubes, channels, or any other suitable pathway through which a sample may flow into the test cell and/or out of the test cell. In one example, the fluid pathway may, at least in part, allow a sample to flow both into and out of the test cell. In another example, the apparatus may include a first fluid pathway and a second fluid pathway, configured to allow fluid to flow into the test cell via one of the first or second fluid pathways and configured to allow fluid to flow out of the test cell via the other of the first or second fluid pathways. The means of loading a sample into the test cell or removing the sample from the test cell may include one or more pumps. The one or more pumps may be configured to draw a sample into the test cell through one or more fluid pathways and then to expel fluid from the test cell through one or more fluid pathways. The one or more pumps may include hydraulic pumps, screw pumps, cavity pumps, peristaltic pumps, impulse pumps, gravity pumps, or any other suitable type of pump. In one particular example, the one or more pumps may include a diaphragm pump. The one or more pumps may draw a sample from a sample tube, reservoir, bottle, or other suitable sample storage means. The sample storage means may itself be part of the apparatus or instead be a distinct and separate component from the apparatus. The means of loading a sample into the test cell or removing the sample from the test cell may include one or more valves. The one or more valves may be arranged to allow or prevent passage of fluid in to and/or out of the test cell. Where the apparatus includes one or more fluid passages through which a sample may pass, the or each fluid passageway may include one or more valves positioned in the or each fluid passageway to allow control of the flow of sample. In an example, a valve may be configured to allow fluid to enter the apparatus when the apparatus is ready to receive and analyse a new sample. In another example, a valve may be configured to hold the sample in the test cell during analysis and then to allow a sample to drain from the apparatus when the apparatus has finished analysing the sample. The one or more valves may be any suitable type of valve including a ball valve, gate valve, globe valve, plug valve, solenoid valve, or any other suitable type of valve. Sample fluid drained from a test cell via one or more fluid pathways and optionally the one or more pumps and/or one or more valves may be directed to waste such as a waste receptacle or drain, or may be directed to one or more further analytical apparatus should additional testing be desired for a given sample. The apparatus may therefore include an inlet, an outlet, a pump, and one or more valves. The inlet, the outlet, the pump and/or the one or more valves may be configured such that the fluid test sample may be loaded into the test cell via the inlet and subsequently drained from the test cell via the outlet

The apparatus or test cell includes a density sensor, a permittivity sensor and a temperature sensor. The density sensor may be any sensor that allows the measurement of the density of a sample in the test cell. In one example, the density sensor may be a balance or scale that measures the mass of the sample which allows the determination of density for a sample or test cell of a fixed volume. In other examples the density sensor may be a flexural resonator, an oscillating tube, a gravity-type sensor, an acoustic-type sensor, a microwave-type sensor, a Coriolis-type sensor, an induced-force type sensor, a MEMS sensor or any other suitable type of density sensor. Where the density sensor is a flexural resonator, the flexural resonator may be an oscillating tube sensor. In this example, the oscillating tube sensor may include a U-shaped tube. The oscillating tube sensor may allow the measurement of the energy lost during oscillating which may, in turn, be used to calculate or determine the viscosity of a fluid. Where both density and viscosity are measured by the density sensor the apparatus may be able to correct the density measurement by taking into account viscosity and any errors the determined viscosity may impart. Depending on the density sensor used in the apparatus, the apparatus may therefore measure dynamic and kinematic viscosity as well as density, temperature and permittivity. The temperature sensor may be any sensor capable of measuring the temperature of the sample contained in the test cell at one or more positions. Suitable temperature sensors include thermocouples, thermometers, thermistors, resistance temperature detectors, semiconductor circuit type devices, or any other suitable temperature sensor. The permittivity sensor may be any sensor that allows measurement of the dielectric permittivity properties of a sample resident in the test cell. Examples of suitable permittivity sensors include capacitance probes, radio frequency type devices, coaxial probe sensors, transmission line sensors, free space microwave sensors, resonant cavity sensors, parallel electrode devices including concentric cylindrical electrode devices and any other suitable permittivity sensor. Where the permittivity sensor is an electrode device, the permittivity sensor may include a plurality of electrodes. The plurality of electrodes may include one or more guard electrodes. In an example, the plurality of electrodes may include a first electrode, a second electrode, a first guard electrode, and a second guard electrode. The guard electrodes may protect the other electrodes in the sensor, aid in the direction of the current flowing through the sample, or promote homogeneity of electric field. The permittivity sensor may further include one or more insulating materials which may prevent leakage of current into unwanted sections of the sensor, test cell or apparatus, and/or limit the passage of heat from or to the permittivity sensor. The permittivity sensor may be configured to measure both permittivity and conductivity of the test sample in the test cell using the plurality of electrodes in the permittivity sensor, where present. The test cell and/or apparatus may include a single permittivity sensor, a single density sensor, and a single temperature sensor. However, the test cell and/or apparatus may include a plurality of one or more of each type of sensor. In an example, the test cell may include a first temperature sensor and the apparatus may include a second temperature sensor, distinct from the test cell. In another example, the test cell may include a plurality of temperature sensors positioned to measure the temperature in different volume regions of the test cell. The skilled person with the benefit of this disclosure will be able to identify a suitable arrangements and configuration of sensors as part of the test cell and/or apparatus. The presence of at least one of each sensor allows the simultaneous measurement of permittivity, density and temperature. Simultaneous measurements are advantageous as it allows the three properties of the sample to be measured at a single moment in time. Small fluctuations in the state of the sample such as variations in temperature may cause significant changes in the density or permittivity of a sample. Measuring all three properties at one time removes the risk of errors introduced by measuring the properties separately on different apparatus as is commonly done in established methods.

The apparatus includes a temperature control means for conditioning the test sample temperature in-situ in the test cell. The temperature control means is operable to increase and/or decrease the temperature of the test cell and/or a sample resident within the internal volume of the test cell. The temperature control means may be configured to maintain the temperature of the test cell and/or a sample residing in the test cell at a constant temperature in use. It is preferable that the temperature control means be a solid-state temperature control means. However, other temperature control may be used without imparting the benefits of a solid-state temperature control means. A solid state-temperature control means may allow for improved precision and control of the temperature of the sample in the test cell while also avoiding other issues commonly associated with fluid-based temperature control. For example, some devices have been known to utilise temperature controlled baths of heptane when obtaining similar measurements which clearly poses health and safety concerns for operators. The solid state temperature control means may include one or more thermoelectric coolers. The one or more thermoelectric coolers may be a Peltier device. Although the term ‘thermoelectric cooler’ indicates that the device is capable of cooling, it should be noted that the term includes devices that are also capable of heating in addition to cooling. The term ‘thermoelectric cooler’ should therefore not be considered limiting to a device that is capable of solely cooling. The apparatus and/or solid state temperature control means may include one or more heat sinks positioned and/or configured to allow heat drawn from the sample using a thermoelectric cooler, or equivalent, to be dispersed to the apparatus' ambient surroundings and/or a fluid flowing across the one or more heat sinks. Where the heat is dispersed to one or more fluids flowing across the one or more heat sinks, a fan, pump, or equivalent may be used to direct fluid across one or more surfaces of the one or more heat sinks.

The apparatus includes a controller. The controller will, in general, be communicably coupled to the density sensor, the permittivity sensor, the temperature sensor and the solid state temperature control means. The controller may be a plurality of components and may, in one example, be in the form of a computer integrated with the apparatus. The controller may be a programmable logic controller (PLC) or other computing device that can carry out instructions. The controller may include one or multiple processing elements that are integrated in a single device or distributed across devices. The controller may have a data input/output interface unit to receive input data from internal or external components or send data to internal or external components. In an example, the controller may have an input device to allow a user to interact with the apparatus. The controller may further include a processor to manage all the components within the controller. Where present, the processor may process all data flow between the components within the controller. The processor may be any of a central processing unit, a semiconductor-based microprocessor, an application specific integrated circuit (ASIC), and/or other device suitable for retrieval and execution of instructions. The controller may further include a storage or memory unit to store any data or instructions which may need to be accessed by, for example, a processor. Where present, the memory unit may be any form of storage device capable of storing executable instructions, such as a non-transient computer readable medium, for example Random Access Memory (RAM), Electrically-Erasable Programmable Read-Only Memory (EEPROM), a storage drive, an optical disc, or the like. In an example, the controller may include a PLC (programmable-logic-controller). In another example, the controller may implement a PID (proportional-integral-derivative) controller. In a further example, the controller may implement a FO (fractional-order) controller. In yet another example, the controller may implement an IO (integer-order) controller. The controller may include circuitry such as measurement circuitry. The controller may operate in a closed-loop or an open-loop manner dependent upon the wider functions to be carried out by the controller. The controller may further include a system model. Where the controller includes a system model, the controller may compare a signal or reading sensed by the density sensor, permittivity sensor and/or temperature sensor with one or a plurality of parameters of the system model.

The apparatus may include one or more additional features in any suitable combination. In one example, the apparatus may include a reservoir in fluid communication with the test cell. The reservoir may be positioned to receive fluid exceeding the volume of the test cell due to thermal expansion of the fluid test sample when resident in the test cell. The reservoir may include an overflow pipe configured to allow excess fluid to flow out of the reservoir. The reservoir and the fluid passageways connecting the test cell to the reservoir may include one or more pumps and/or valves to allow the flow of fluid to the reservoir or from the reservoir to be controlled. The test cell, reservoir, or any combination thereof may include one or more level sensors configured to detect the level of fluid in the test cell and/or reservoir. The level sensors may include a maximum level sensor and a minimum level sensor configured to determine when the test cell is filled and/or when the test cell is at the minimum acceptable operating volume of fluid. The level sensor may be a dynamic level sensor configured to detect the volume of fluid in the test cell and/or reservoir between a maximum and minimum level. In an example, the test cell may include a maximum level sensor and a minimum level sensor. In another example, the reservoir may include a maximum level sensor and a minimum level sensor. In a yet further example, both the test cell and the reservoir may each include a maximum and a minimum level sensor. The controller may be configured to actuate one or more valves of the apparatus and/or operate at least one of the one or more pumps in response to a signal from a level sensor. The controller may actuate one or more valves and/or operate one or more pumps to increase the volume of fluid in the test cell, increase the volume of fluid in the reservoir, decrease the volume of fluid in the test cell, decrease the volume of fluid in the reservoir, or any combination thereof. The level sensor may be configured to detect an insufficient sample volume in the test cell and the controller may be configured, based upon a signal from the level sensor, to correct one or more signals, readings, or the like received from one or more further sensors forming part of the apparatus based on the level sensor signal.

The apparatus may further include a display. The controller may be configured to output data such as the result of one or more calculations, one or more readings of the density sensor, permittivity sensor, and/or temperature sensor, or one or more graphical representations of data to the display. The controller may therefore be configured such that one or more signals may be sent to the display which cause the display to output information relating to the permittivity, relative permittivity, density, temperature, or any combination thereof of the fluid test sample. The apparatus may further include one or more data conduits such as an ethernet connection or wireless device that may allow data to be transmitted to one or more distant or proximate locations and/or devices from the apparatus. In an example, the apparatus may transmit data to one or more computers, data storage devices, servers, or the like. The apparatus may include a housing. The housing may, at least in part, include or be formed from one or more thermally insulating materials. The thermally insulating housing may house, encompass or otherwise contain at least part of the test cell, temperature control means, and/or the controller. The apparatus may include a sampling system such as an autosampler or be configured such that it may be coupled to a separate autosampling device as required.

The apparatus may be wholly or partly automated using the controller. In general, any component coupled to the controller may be automated such that the component functions without direct interaction of the user or with limited interaction from a user. Automation of the apparatus may reduce the operator time required to suitably analyse a given sample. The controller may be configured to automatically adjust the temperature of the test cell towards a predetermined value in response to a signal from the density sensor, the permittivity sensor and/or the temperature sensor. The controller may automatically adjust the temperature by sending a signal to the temperature control means. Where the temperature control means is a solid state temperature control means, the controller may be configured to automatically adjust the temperature by sending one or more signals to the solid state temperature control means. The controller may also perform one or more calculations. The controller may store instructions which, when executed, cause the controller to automatically calculate the relative permittivity of a sample in a test cell, the relationship between permittivity and density, or any combination thereof based on a signal from the density sensor, the permittivity sensor and/or the temperature sensor. The controller may be configured to calibrate the permittivity sensor, density sensor and/or temperature sensor based upon one or more readings obtained from a calibration reference sample and/or blank sample loaded into the test cell. The permittivity sensor may be calibrated using a vacuum, a gas such as helium or nitrogen, a reference liquid, or any combination thereof. The controller may automatically identify the presence of a calibration reference sample and/or blank automatically based upon signals obtained from a sensor or other component of the apparatus, or may identify the presence of such samples based upon user input. Where a sensor requires a calculation to determine a parameter, the controller may also perform such calculations. In one example, where the density sensor includes a scale or balance, the controller may determine the density based upon a signal from the balance or scale and a known volume of sample in the test cell. In another example, where the density sensor determines density and viscosity, the controller may correct the density for errors imparted due to the viscosity of the sample. Where the apparatus includes one or more additional temperature sensors, such sensors may be communicably coupled to the controller the controller may be configured to determine a temperature gradient in the test cell based upon a signal from the temperature sensor and the one or more additional temperature sensors. The controller may automatically adjust the temperature of the test cell towards a predetermined value in response the determination of a temperature gradient. The controller may store instructions which, when executed, cause the controller to extrapolate permittivity, relative permittivity, density, temperature, or any combination thereof based upon signals from the density sensor, the permittivity sensor, the temperature sensor and/or any other sensors communicably coupled to the controller. Where one or more pumps and/or one or more valves are present, the one or more pumps and/or one or more valves may be communicably coupled to the controller. In this configuration, the controller may be configured to automatically load a sample into the test cell and/or drain a sample from the test cell by operating the one or more pumps and/or the one or more valves by sending a signal to the pump and/or the one or more valves. Where the apparatus includes or is coupled to an autosampler, the controller may be communicably coupled to the autosampler such that a plurality of different samples contained in the autosampler may be loaded, analysed and discarded in succession. The controller may additionally, or alternatively, be configured such that the one or more pumps will meter a fixed volume of sample into the test cell. The skilled person will appreciate that the modes of automation described may be combined in any suitable manner so as to reduce analysis and processing time and to reduce the burden of interaction placed upon a user.

FIG. 1 shows an analytical apparatus within the scope of the present invention. The apparatus includes test cell 1 in which sample fluid may be received. The test cell 1 includes a permittivity sensor 4, a density sensor 5, and a sample temperature sensor 6. Each sensor 4,5,6 is positioned such that they ‘see’ the density, temperature and permittivity, respectively, of the sample within the test cell 1. The test cell further includes a first additional sensor position 15 and a second additional sensor position 16. The first and second additional sensor positions 15,16 are ports into which one or more additional sensors may be inserted. The first and second additional sensor positions 15,16 may be shaped to fit a type of sensor as desired by a user. In the example shown in FIG. 1, the first and second additional sensor positions 15,16 are shaped to receive a further test cell temperature sensor or a temperature verification thermometer (not shown). The test cell 1 is fluidly coupled with a sample source via fluid inlet 12. An inlet valve 9 is positioned along the fluid inlet 12 such that the inlet valve may allow passage of fluid, restrict the passage of fluid or wholly prevent the passage of fluid through fluid inlet 12. A pump 14 is positioned at the end of fluid inlet 12 at the end of the fluid inlet 12 proximate to the test cell 1. The fluid inlet 12 may optionally include a filter 26. The filter 26 may be configured to remove particles of a desired size or greater from the fluid being drawn into the apparatus through the fluid inlet 12. The filter 26 may be selected to allow particles of a desired size range to enter the apparatus. The test cell 1 is also fluidly coupled to a waste receptacle via fluid outlet 13. An outlet valve 8 is positioned along the fluid outlet 13 such that the outlet valve 8 may allow passage of fluid, restrict the passage of fluid or wholly prevent the passage of fluid through fluid outlet 13. The pump 14 is positioned such that it may also direct fluid from the test cell through the fluid outlet 13 towards the waste receptacle. The test cell 1 is also fluidly coupled to an expansion reservoir 10. The expansion reservoir 10 is positioned at the end of the test cell 1 distant from the pump 14 such that fluid exceeding the volume of the test cell may flow into the expansion reservoir 10. An overflow pipe 11 is connected to the fluid reservoir 10 to allow any fluid exceeding the volume of the expansion reservoir 10 to flow to drain.

Thermoelectric coolers 7 are positioned on either side of the test cell 1. The thermoelectric coolers 7 are coupled to heatsinks 3 such that heat drawn from the test cell 1 by the thermoelectric coolers 7 may be dissipated. Although two thermoelectric coolers 7 and heatsinks 3 are shown in FIG. 1, the skilled person will appreciate that thermoelectric coolers 7 and heatsinks 3 may be positioned on each of the four sides of the test cell 1 or only on one or more sides of the test cell 1 as desired. The test cell 1 and thermoelectric coolers 7 are positioned inside thermally insulated housing 2 with apertures provided in the housing 2 such that the thermoelectric coolers 7 connect with the heat sinks 3 and such that the test cell 1 connects with the temperature sensor 6, permittivity sensor 4, density sensor 5 and additional temperature sensors positioned in the first and/or second additional sensor positions 15,16 where present. The apparatus of FIG. 1 further includes a controller 18 for automating operation of the apparatus and performing calculations, and display 19 to which the controller 18 may output information for presentation to a user.

In use, when a sample is to be drawn into the internal volume of the test cell 1, inlet valve 9 is opened to place the test cell 1 in fluid communication with the sample source. Pump 14 is then operated to draw the fluid from the sample source through fluid inlet 12 into the internal volume of the test cell 1. Once a sample has been loaded into test cell 1, the inlet valve 9 is closed and operation of the pump is stopped. A sample is consequently retained within the internal volume of test cell 1. Any fluid pumped into the test cell 1 via the pump 14 that exceeds the volume of test cell 1 will flow into the expansion reservoir 10. Should fluid in excess of the volume of the test cell 1 continue to flow into the expansion reservoir 10 due to a fault with the pump 14, inlet valve 9 or other rationale, the fluid exceeding the volume of the expansion reservoir 10 will flow out of the expansion reservoir 10 via the overflow pipe 11. Once a sample has been loaded into the test cell 1, the permittivity sensor 4, the density sensor 5, and the sample temperature sensor 6 are operated via the controller 18 to determine the permittivity, density and temperature of the sample. The sensors 4, 5, 6 may be used to measure parameters individually or simultaneously depending on the instructions stored in, and executed by, the controller 18. The sensors 4,5,6 send signals to the controller 18 which then outputs one or more parameters of the sample via the display 19. The controller 18 may also calculate one or more further parameters such as the absolute permittivity of the sample and output this value via the display 19. If the temperature of the sample in the test cell 1 is not at the temperature at which the sample is to be measured, or if a range of measurements at different temperatures are required, the controller 18 will send signals to the thermoelectric coolers 7 to cause a change in temperature of the sample in the test cell 1. Multiple measurements of one or multiple parameters may be performed on each sample. Once all measurements are complete, the controller 18 may open the outlet valve 8 to allow the sample in the test cell 1 to drain to waste through outlet 13. Depending on the configuration of the apparatus and/or the nature of the sample, the controller 18 may also operate the pump 14 to aid in draining the sample from the internal volume of test cell 1. The user may also select an option on an input device (not shown) to manually drain the sample from the test cell. Once the sample has been suitably drained from the test cell 1, a new sample may be loaded and analysed.

The apparatus of the present invention, including the apparatus shown and described in relation to FIG. 1, may be used with one or more test methods used to determine density, permittivity, temperature, and/or the level of dispersed particles in a sample. One test method with which the apparatus of the present invention may be used is the “IP PM FC/21: Determination of Relative Permittivity (Dielectric Constant) of Aviation Turbine Fuel, Small Scale Automated Temperature Scanning Method” available from the Energy Institute. The methods with which the apparatus of the invention may be used may involve (i) filling a permittivity sensor consisting of an inner electrode, outer electrode and guard electrode, with a test sample; (ii) measuring capacitance of the sample throughout a range of temperatures selected or determined by a user at a known excitation frequency; (iii) calculating the relative permittivity of the sample; and optionally (iv) repeating steps (i) to (iii) at further temperatures across a range to determine the change in relative permittivity with changes in temperature. The method may also include: (v) measurement of the density of the sample at each temperature at which permittivity is measured; and (vi) determining the relationship between density and permittivity.

The methods of using the apparatus of the present invention may include waiting until the temperature of a sample has been stable for a given period prior to measuring permittivity and/or density. In some examples, the methods may involve waiting up to 1 minute, up to 2 minutes, up to 3 minutes, up to 5 minutes, up to 10 minutes, up to 15 minutes, up to 20 minutes, or more for the temperature of the sample to be stable. A stable temperature may be a temperature that has changed by less than or equal to 0.01° C., less than or equal to 0.05° C., less than or equal to 0.1° C., less than or equal to 0.2° C., less than or equal to 0.3° C., less than or equal to 0.4° C., less than or equal to 0.5° C. or less than or equal to 1° C. However, the methods of using the apparatus of the present invention may not include waiting until the temperature of a sample has been stable for a given period of time. Such a method may be advantageous as it may reduce the time required to obtain one or more measurements using the apparatus of the invention. The methods of using the apparatus of the present invention may include determining a constant for the test cell using a standardising fluid. The standardising fluid may be any suitable fluid, certified reference material, or the like whose properties are sufficiently known such that one or more constants of the test cell and/or wider apparatus may be determined via use of the fluid. The fluid may be selected depending upon the temperatures and/or temperature ranges at which the apparatus is to be operated. In one example, the standardising fluid may be cyclohexane. In other examples where lower temperatures such as −40° C. are to be used, the standardising fluid may be toluene. The skilled person, with the benefit of this disclosure, will be able to select a suitable standardising fluid for use with the apparatus and associated methods.

FIG. 2 shows an example of a permittivity sensor that may be used with apparatus of the present invention such as FIG. 1. The permittivity sensor of FIG. 2 includes an inner electrode 22 and an outer electrode 23 to allow measurement of permittivity. The sensor further includes upper guard electrode 20 and lower guard electrode 25 in addition to various insolating components including upper insulator 21, lower insulator 24 and insulators 26.

The integrated electromechanical apparatus of FIG. 1 may be miniaturised in scale or may be of a larger size suitable for use on a laboratory bench or equivalent. Where the apparatus is miniaturized, the apparatus may contain a miniaturised test cell, miniaturised sensors, a miniaturised controller, miniaturised temperature control means, and a miniaturised display. In an example, a miniaturised apparatus may be of dimensions 300 mm×200 mm×200 mm. In another example, a miniaturised apparatus may be of dimensions 380 mm×260 mm×260 mm. In a further example, a miniature apparatus may be of dimensions 420 mm×300 mm×300 mm.

The apparatus of the invention, such as an apparatus as shown in FIG. 1 may interface with a computer and storage means to record the data derived from the apparatus automatically without user intervention. In an example, a user may setup one or more samples at the beginning of an analysis job and instruct the apparatus and computer via an interface to analyse all the samples setup by the user. Once instructed, the apparatus may measure all samples and record the required information without further interaction from a user. Where rapid testing is desired, the controller and/or the computer may use extrapolation calculations to adjust the permittivity and density results before the test cell is thermally stable. Such extrapolation techniques may reduce individual sample analysis time and consequently speed up an analysis job that includes multiple samples. The controller and/or a computer interfaced with the apparatus may automatically plot and display plotted information as an analysis job progresses or at the end of the analysis job.

In some applications, the apparatus of the invention may be used in an in-line and/or on-line manner. For example, the apparatus could form part of, or be positioned in proximity to, a fluid vessel such as a storage medium or flow pathway. Examples of fluid vessels include a pipeline, fuel-line, tank, tank farm, fuel bowser, or the like. The apparatus may be attached to the fluid vessel to allow for continuous or continual monitoring of fuel stored in, or being directed out of, the fluid vessel in question. In one example, the apparatus may be integral and/or embedded in the fluid vessel. In another example, the apparatus may be in proximity to the fluid vessel with samples drawn from the fluid vessel to the apparatus and then returned to the fluid vessel once analysis is complete. In one particular example, the apparatus may be used to monitor the dielectric characteristics of fuel that is being loaded into an aircraft.

The apparatus may be used in one or more additional ways. In one example, the test cell may measure, or allow the determination of, the conductivity of a sample. The test cell as described herein allows measurement of permittivity. Conductivity and complex permittivity are mathematically related and therefore a measurement of complex permittivity by the test cell may be used to determine conductivity. In this manner, the conductivity may be measured by measurement of the complex permittivity of a sample. The determination of conductivity may be performed via a controller and/or computer interfaced with the apparatus and/or test cell. The determination of conductivity may be carried out using a mathematical transformation.

The density sensor of the apparatus may be used to measure and/or estimate the viscosity of a sample. However, in other examples, the apparatus may include a viscosity measurement device. The viscosity measurement device may be a viscometer. The viscometer may be an aperture or conduit flow viscometer, a vibrational viscometer, or a rotational viscometer. In a particular example, the viscometer is a rotational viscometer. Where the apparatus includes a viscosity measurement device, the viscosity measurement device may form part of the test cell. In other examples, a sample may be introduced to a viscosity measurement device that forms part of the apparatus but is separate from the test cell. The viscosity measurement device may therefore include a chamber including a rotor and a means of measuring rotational speed. In other examples, the viscosity measurement device may include components configured to allow at least a portion of a sample to flow under gravity. If the viscosity measurement device is configured to measure dynamic viscosity, then the kinematic viscosity of a sample may be calculated. The calculation may be performed by a controller and/or computer interfaced with the apparatus. In examples where at least a portion of a sample is allowed to flow under gravity, the time taken for a fixed volume to flow may be determined by one or more sensors and then the kinematic viscosity may be determined by the apparatus. For the avoidance of doubt, any property, characteristic, or other value relating to a sample measured, determined, and/or calculated by the apparatus may be passed to a display such that the information may be presented to a user. The skilled person, with the benefit of this disclosure, will also appreciate that the apparatus may measure viscosity and/or kinematic viscosity using a viscosity measurement device and then verify the measurement via a determination of viscosity carried out via the density sensor or vice versa.

The apparatus described herein are therefore well suited to application in various industries. The apparatus enables the user to safely and easily measure and record the permittivity, volumetric mass density and temperature of a sample simultaneously. In some examples, the apparatus may allow a user to measure or determine each of the density, permittivity, temperature, conductivity, and viscosity of a sample. The apparatus further enables the user to measure the permittivity and volumetric mass density at various temperatures without user intervention. The controller and/or computer interfaced with the apparatus may automatically calculate the results desired by the user from the stored calibrations and vacuum permittivity. The apparatus therefore saves time, reduces cost, and enables users to characterise the relationship between the permittivity of a sample and its volumetric mass density at a specific known temperature or a series of known temperatures. The user is enabled to do these measurements within a self-contained apparatus which may have a reduced spatial footprint when compared to the apparatus required for conventional methods of determining the permittivity and density relationship of dielectric samples. Built-in solid state thermal heating and cooling enables fast manipulation of the temperature and quick test results. No additional equipment, such as environmental chambers, baths or ovens are required to use the apparatus. The measurements can be done on a small sample by a non-scientific operator. These and other advantages will be apparent to the skilled person with the benefit of this disclosure. The scope of the invention is defined by the appended claims.

1. An apparatus for simultaneously measuring permittivity, density and temperature of a fluid test sample, the apparatus comprising:
a solid state temperature control means for conditioning the fluid test sample temperature in-situ;
a test cell for receiving the fluid test sample, the test cell comprising:
a density sensor
a permittivity sensor; and
a temperature sensor; and
a controller communicably coupled to the density sensor, the permittivity sensor, the temperature sensor and the solid state temperature control means.

2. An apparatus according to claim 1, wherein the solid state temperature control means comprises one or more thermoelectric coolers.

3. An apparatus according to claim 1, wherein the solid state temperature control means comprises one or more heat sinks.

4. An apparatus according to claim 1, wherein the controller is configured to automatically adjust the temperature of the test cell towards a predetermined value in response to a signal from the density sensor, the permittivity sensor and/or the temperature sensor.

5. An apparatus according to claim 4, wherein the controller is configured to automatically adjust the temperature by sending one or more signals to the solid state temperature control means.

6. An apparatus according to claim 1, wherein the apparatus further comprises a reservoir in fluid communication with the test cell, the reservoir positioned to receive fluid exceeding the volume of the test cell due to thermal expansion of the fluid test sample when resident in the test cell.

7. An apparatus according to claim 6, wherein the reservoir comprises an overflow pipe configured to allow excess fluid to flow out of the reservoir.

8. An apparatus according to claim 1, wherein the apparatus further comprises:
an inlet, wherein the inlet optionally comprises a filter;
an outlet;
a pump; and
one or more valves;
wherein the inlet, the outlet, the pump and/or the one or more valves are configured such that the fluid test sample may be loaded into the test cell via the inlet and subsequently drained from the test cell via the outlet.

9. An apparatus according to claim 8, wherein:
the controller is communicably coupled to the pump and/or the one or more valves; and
the controller is configured to automatically load the fluid test sample into the test cell and subsequently drain the fluid test sample from the test cell by operating the pump
and/or the one or more valves by sending a signal to the pump and/or the one or more valves.

10. An apparatus according to claim 9, wherein the apparatus further comprises one or more level sensors, and the controller is configured to actuate the one or more valves and/or operate the pump in response to a signal from the one or more level sensors.

11. An apparatus according to claim 1, wherein the controller stores instructions which, when executed, cause the controller to automatically calculate the relative permittivity of a sample in a test cell, the relationship between permittivity and density, or any combination thereof based on a signal from the density sensor, the permittivity sensor and/or the temperature sensor.

12. An apparatus according to claim 1, the apparatus further comprising:
one or more additional density sensors, permittivity sensors, and/or temperature sensors; and/or one or more viscosity measurement devices.

13. An apparatus according to claim 12, wherein:
the apparatus comprises one or more additional temperature sensors and the one or more additional temperature sensors are communicably coupled to the controller, and
the controller is configured to determine a temperature gradient in the test cell based upon a signal from the temperature sensor and the one or more additional temperature sensors and to automatically adjust the temperature of the test cell towards a predetermined value in response the determination of the temperature gradient.

14. An apparatus according to claim 1, wherein the controller stores instructions which, when executed, cause the controller to extrapolate permittivity, relative permittivity, density, temperature, or any combination thereof based upon signals from the density sensor, the permittivity sensor, the temperature sensor and/or any other sensors communicably coupled to the controller.

15. An apparatus according to claim 1, wherein:
the apparatus further comprises a display;
the controller is communicably coupled to the display; and
the controller is configured such that one or more signals may be sent to the display which cause the display to output information relating to the permittivity, relative permittivity, density, temperature, conductivity, viscosity, or any combination thereof of the fluid test sample.

16. An apparatus according to claim 1, wherein:
the apparatus comprises a thermally insulated housing; and
the solid state temperature control means and/or test cell are at least partially positioned inside the thermally insulated housing.

17. An apparatus according to claim 1, wherein the permittivity sensor comprises plurality of electrodes, the plurality of electrodes comprising a first electrode and a second electrode.

18. An apparatus according to claim 17, wherein the plurality of electrodes further comprises a first guard electrode and a second guard electrode.

19. An apparatus according to claim 17, wherein the permittivity sensor comprised one or more insulating materials.

20. An apparatus according to claim 1, wherein the density sensor comprises a flexural resonator.

21. An apparatus according to claim 20, wherein the flexural resonator comprises one or more U-shaped tubes.

22. An apparatus according to claim 20, wherein the flexural resonator comprises a MEMS device.

23. An apparatus according to claim 21, wherein the flexural resonator is configured to measure viscosity of the fluid test sample based upon the energy lost during oscillation.

24. An apparatus according to claim 23, wherein the controller is configured to correct a density measurement from the density sensor based upon the measurement of viscosity.

25. An apparatus according to claim 1, wherein the permittivity sensor is configured to measure the conductivity of a fluid test sample, wherein the conductivity is measured simultaneously alongside a simultaneous measurement of density, permittivity and temperature.

26. An apparatus according to claim 1, wherein the apparatus is in-line with, or in proximity to, a fluid vessel and configured for continuous or continual simultaneous measurement of permittivity, density and temperature of at least a portion of fluid in the fluid vessel.

27. An apparatus according to claim 26, wherein the fluid vessel is a pipeline, a fuel-line, a tank, a tank farm, or a fuel bowser,
